(19) United States
(12) Patent Application Publication
(10) Pub. No.: US 2023/0210377 A1
(43) Pub. Date: Jul. 6, 2023

(54) DEEP FLUORESCENCE IMAGING BY LASER-SCANNING EXCITATION AND ARTIFICIAL NEURAL NETWORK PROCESSING

(71) Applicant: National University of Singapore, Singapore (SG)

(72) Inventors: Zhi Kuang Tan, Singapore (SG); Daryl Darwan, Singapore (SG); Kang Rui Garrick Lim, Singapore (SG); Hadhi Wijaya, Singapore (SG)

(73) Assignee: National University of Singapore, Singapore (SG)

(21) Appl. No.: 18/000,784

(22) PCT Filed: Jun. 4, 2021
(86) PCT No.: PCT/SG2021/050321

§ 371 Date: Dec. 5, 2022

(30) Foreign Application Priority Data
Jun. 5, 2020 (SG) 10202005338X

(51) Int. Cl. A61B 5/00; G06T 3/40; A61K 49/00; G06N 3/084; G01N 21/64

(52) U.S. Cl. CPC A61B 5/0071; G06T 3/4076; A61K 49/0019; A61K 49/0093; A61B 5/7264; G06N 3/084; G01N 21/6458; A61B 2503/40

(57) ABSTRACT

The current invention relates to the use of a neural network to improve the quality of images obtained from light scattered by an intermediate object that scatters light, such as tissue or a frosted screen. The invention relates to a method of imaging a human or animal bode using a nanocrystal array capable of fluorescing upon excitation from light from a near-infrared light source. This invention also relates to detection means and apparatus used in said methods, as well as to quantum dots useful in said use.

### FIELD OF INVENTION

The current invention relates to the use of a neural network to improve the quality of images obtained from light scattered by an intermediate object that scatters light, such as tissue or a frosted screen. This invention also relates to detection means and apparatus used in said methods, as well as to quantum dots useful in said use.

### BACKGROUND

The listing or discussion of a prior-published document in this specification should not necessarily be taken as an acknowledgement that the document is part of the state of the art or is common general knowledge.

The development of targeted therapy or immunotherapy drugs for cancer have typically relied on the identification of antibodies that could inhibit cancer cell growth, deliver payload of drugs to targeted cells, or to activate a targeted immune response on the cancer cells, thereby killing cancer cells without causing damage to healthy cells. Visible fluorescent probes have served as the workhorse in the in vitro and in vivo testing of drug efficacy, since the detection of fluorescent-tagged antibodies around cancer tissues, typically in animal tests, would indicate the successful development of an antibody that is specific in targeting the cancer cells.

However, existing fluorescence imaging methods have significant shortcomings, where both the excitation radiation and the fluorescence signal have difficulties penetrating deep into and out of biological tissues due to the intense absorption and scattering of visible light by the skin, tissues and biological fluids (Weissleder, R., Nat. Biotechnol. 2001, 19, 316-317; and Frangioni, J. V., Curr. Opin. Chem. Biol. 2003, 7, 626-634). Hence, such fluorescence testing methods have been limited to the use of nude mice, which are typically smaller than the size of a child's palm. However, murine (mice) models for cancer immunotherapy studies are vastly inadequate due to the significant differences between human and mice immunology, hence leading to high failure rates in the translation of preclinical studies in mice to clinical trials in humans. The ability to probe cancer drug efficacy in larger animals, such as rabbits or pigs, that possess greater homology with humans could therefore be valuable in cancer drug developments. Nonetheless, due to the depth limitations of existing fluorescence imaging methods, the detection of deeper cancers in larger animals have required magnetic resonance imaging (MRI) or positron emission tomography (PET) techniques, which are prohibitively expensive for cancer drug screening.

Therefore, there is an urgent need for an alternative deep imaging technique that could be deployed, at low cost, for immunotherapy studies and targeted drug development.

There is also a need for improved non-invasive imaging devices and techniques.

### SUMMARY OF INVENTION

Aspects and embodiments of the current invention are provided in the following numbered clauses.

1. A computerized method for processing scattered images obtained by imaging through scattering media, comprising:
providing a trained neural network model trained with a training dataset of scattered images comprising associated pairs of low-resolution images and high-resolution images, each image comprising a series of separated bands;
receiving an input scattered image by the trained neural network model;
processing the input scattered image using the trained neural network model; and
generating an output image by the trained neural network model in response to said processing of the input scattered image,
wherein the output image has a higher resolution than the input scattered image.
   2. The computerized method according to clause 1, further comprising training the neural network model, said training comprising:
(a) extracting, from the training dataset, an associated pair of low-resolution and high-resolution images;
(b) identifying an input pixel from the low-resolution image and a corresponding true output pixel from the high-resolution image;
(c) selecting a cluster of pixels from the low-resolution image, the cluster of pixels surrounding the input pixel;
(d) weighting each pixel in a cluster of pixels using a set of weight and bias parameters;
(e) generating a processed output pixel from said processing of the cluster of pixels;
(f) determining an error between the processed output pixel and the true output pixel;
(g) backpropagating the error to adjust the parameters in step (d);
(h) iteratively performing steps (d) to (g) to minimize the error,
wherein the minimized error is associated with an optimized set of parameters for the neural network model.
   3. The computerized method according to clause 2, said training further comprising repeating steps (a) to (h) for each associated pair of images in the training dataset.
   4. The computerized method according to clause 2 or 3, wherein step (d) comprises successively weighting each pixel at least twice.
   5. The computerized method according to any one of clauses 2 to 4, wherein step (e) comprises:
generating a raw output pixel from said processing of the cluster of pixels;
processing the raw output pixel using a logistic function; and
generating the processed output pixel from said processing of the raw output pixel.
   6. The computerized method according to any one of clauses 2 to 5, wherein step (g) is performed using a gradient descent function.
   7. The computerized method according to any one of clauses 2 to 6, wherein the error comprises a mean squared error.
   8. The computerized method according to any one of clauses 2 to 7, wherein the scattered images are fluorescence images.
   9. A method of imaging a part or the whole of a human or animal body, using an imaging device, comprising:
a near-infrared light source;
a light directing means or apparatus;
an array comprising nanocrystals (e.g. giant shell quantum dots) capable of fluorescing upon excitation from light from the near-infrared light source; and
a detecting means or apparatus configured to detect light emitted by the nanocrystals, where the method comprises the steps of:
   (a) positioning a first side of the part or whole of the human or animal body to be imaged to face the near-infrared light source, light directing means or apparatus and a detecting means or apparatus and a second side of the part or whole of the human or animal body to be imaged to face an array comprising nanocrystals;
   (b) directing near-infrared light from the near-infrared light source through the first and second surfaces of the part or whole of the human or animal body to be imaged via the light directing means or apparatus and into the panel comprising nanocrystals; and
   (c) detecting fluorescent light released from the nanocrystals using the detecting means or apparatus.
   10. The method according to Clause 9, further comprising the step of capturing an image of the part or whole of the human or animal body to be imaged based on the detected fluorescent light, the image being a scattered image.
   11. The method according to Clause 10, further comprising the step of processing the scattered image using the computerized method described in any one of Clauses 1 to 8 to enhance the scattered image.
   12. The method according to any one of Clauses 9 to 11, wherein, in the imaging device:
   (a) the near-infrared light source is a laser capable of emitting light at near-infrared wavelengths; and/or
   (b) the light directing means or apparatus comprises a mirror; and/or
   (c) the array comprising nanocrystals is positioned on a moveable platform such that the array is movable relative to the near-infrared light source and/or a light beam from the near-infrared light source is moveable relative to the array; and/or
   (d) the detecting means or apparatus further comprises an imaging apparatus, optionally wherein the detecting means or apparatus further comprises an imaging processing unit.
   13. The method according to any one of Clauses 9 to 11, wherein, in the imaging device the nanocrystals are capable of fluorescing upon excitation from light from the near-infrared light source are giant shell quantum dots having the formula:

In(Zn)As—In(Zn)P—GaP—ZnS

wherein:
In(Zn)As is the core of the quantum dot;
In(Zn)P is the giant shell;
GaP represents an interlayer shell between In(Zn)P and ZnS; and
   ZnS represents an outer layer shell of the quantum dot.
   14. The method according to Clause 13, wherein the quantum dot is one in which one or more of the following apply:
   (a) the ZnS outer layer comprises ZnS and a hydrophobic or a hydrophilic organic compound, optionally wherein the hydrophobic organic compound is oleic acid, optionally wherein the hydrophilic organic compound is mercaptosuccinic acid, further optionally wherein the hydrophilic organic compound is functionalised with a biological targeting agent (e.g. the biological targeting agent may be selected from one or more of the group consisting of folic acid and a cancer-specific antibody);
   (b) the quantum dot displays an emission peak at from 820 to 850 nm, such as from 828 to 837 nm, such as 828 nm or 837 nm; and/or
the quantum dot displays a photoluminescence lifetime of from 20 to 100 ns, such as from 30 to 70 ns, such as from 40 to 60 ns, such as 59 ns; and/or
the quantum dot absorbs light at a wavelength of from 400 to 800 nm; and/or
the quantum dot displays a photoluminescence quantum efficiency of from 60 to 75%;
   (c) the quantum dot has an average size according to transmission electron microscopy of from 6 to 7 nm, such as 6.6 nm; and/or
the quantum dot has an average hydrodynamic size of from 8 to 9 nm, such as 8.6 nm;

and

(d) the atomic percentages in the quantum dot are as follows: In from 35 to 45%; As from 1 to 5%; P from 25 to 35%; Zn from 5 to 10%; Ga from 5 to 9%; and S from 8 to 15%, optionally wherein the atomic percentages in the quantum dot are as follows: In from 39.7 to 39.8%; As from 2.2 to 2.3%; P from 31.3 to 31.4%; Zn from 8.5 to 8.6%; Ga from 7.5 to 7.6%; and S from 10.3 to 10.4%.
15. An imaging device comprising:
a near-infrared light source;
a light directing means or apparatus;
an array comprising nanocrystals (e.g. giant shell quantum dots) that are capable of
fluorescing upon excitation from light from the near-infrared light source; and
a detecting means or apparatus configured to detect light emitted by the nanocrystals.
   16. The imaging device according to Clause 15, wherein:
   (a) the near-infrared light source is a laser capable of emitting light at near-infrared wavelengths; and/or
   (b) the light directing means of apparatus comprises a mirror; and/or
   (c) the array comprising nanocrystals is positioned on a moveable platform.
   17. The imaging device according to Clause 15 or Clause 16, wherein the detecting means or apparatus further comprises an imaging apparatus, optionally wherein the detecting means or apparatus further comprises an imaging processing unit.
   18. The imaging device according to any one of Clauses 15 to 17, wherein, in the imaging device the nanocrystals capable of fluorescing upon excitation from light from the near-infrared light source are giant shell quantum dots having the formula:

In(Zn)As—In(Zn)P—GaP—ZnS

wherein:
In(Zn)As is the core of the quantum dot;
In(Zn)P is the giant shell;
GaP represents an interlayer shell between In(Zn)P and ZnS; and
   ZnS represents an outer layer shell of the quantum dot.
   19. The imaging device according to Clause 18, wherein the quantum dot is one in which one or more of the following apply:
   (a) the ZnS outer layer comprises ZnS and a hydrophobic or a hydrophilic organic compound, optionally wherein the hydrophobic organic compound is oleic acid, optionally wherein the hydrophilic organic compound is mercaptosuccinic acid, further optionally wherein the hydrophilic organic compound is functionalised with a biological targeting agent (e.g. the biological targeting agent may be selected from one or more of the group consisting of folic acid and a cancer-specific antibody);
   (b) the quantum dot displays an emission peak at from 820 to 850 nm, such as from 828 to 837 nm, such as 828 nm or 837 nm; and/or
the quantum dot displays a photoluminescence lifetime of from 20 to 100 ns, such as from 30 to 70 ns, such as from 40 to 60 ns, such as 59 ns; and/or
the quantum dot absorbs light at a wavelength of from 400 to 800 nm; and/or
the quantum dot displays a photoluminescence quantum efficiency of from 60 to 75%;
   (c) the quantum dot has an average size according to transmission electron microscopy of from 6 to 7 nm, such as 6.6 nm; and/or
the quantum dot has an average hydrodynamic size of from 8 to 9 nm, such as 8.6 nm; and
   (d) the atomic percentages in the quantum dot are as follows: In from 35 to 45%; As from 1 to 5%; P from 25 to 35%; Zn from 5 to 10%; Ga from 5 to 9%; and S from 8 to 15%, optionally wherein the atomic percentages in the quantum dot are as follows: In from 39.7 to 39.8%; As from 2.2 to 2.3%; P from 31.3 to 31.4%; Zn from 8.5 to 8.6%; Ga from 7.5 to 7.6%; and S from 10.3 to 10.4%.
   20. A method of diagnosis comprising the steps of:
   (a) supplying a plurality of nanocrystals (e.g. giant shell quantum dots) capable of fluorescing upon excitation from light from the near-infrared light source to a subject;
   (b) subjecting a target site on the subject to light irradiation; and
   (c) detecting a signal, or lack thereof, generated by the quantum dots to provide a diagnosis.
   21. The method according to Clause 20, further comprising the step of capturing an image of the target site based on the detected signal, the image being a scattered image.
   22. The method according to Clause 20 or Clause 21, further comprising the step of processing the scattered image using the computerized method described in any one of Clauses 1 to 8 to enhance the scattered image for the diagnosis.
   23. The method according to any one of Clauses 20 to 22, wherein, in the imaging device the nanocrystals are giant shell quantum dots capable of fluorescing upon excitation from light from the near-infrared light source are a giant shell quantum dot having the formula:

In(Zn)As—In(Zn)P—GaP—ZnS

wherein:
In(Zn)As is the core of the quantum dot;
In(Zn)P is the giant shell;
GaP represents an interlayer shell between In(Zn)P and ZnS; and
   ZnS represents an outer layer shell of the quantum dot.
   24. The method according to Clause 23, wherein the quantum dot is one in which one or more of the following apply:
   (a) the ZnS outer layer comprises ZnS and a hydrophobic or a hydrophilic organic compound, optionally wherein the hydrophobic organic compound is oleic acid, optionally wherein the hydrophilic organic compound is mercaptosuccinic acid, further optionally wherein the hydrophilic organic compound is functionalised with a biological targeting agent (e.g. the biological targeting agent may be selected from one or more of the group consisting of folic acid and a cancer-specific antibody);
   (b) the quantum dot displays an emission peak at from 820 to 850 nm, such as from 828 to 837 nm, such as 828 nm or 837 nm; and/or
the quantum dot displays a photoluminescence lifetime of from 20 to 100 ns, such as from 30 to 70 ns, such as from 40 to 60 ns, such as 59 ns; and/or
the quantum dot absorbs light at a wavelength of from 400 to 800 nm; and/or
the quantum dot displays a photoluminescence quantum efficiency of from 60 to 75%;
   (c) the quantum dot has an average size according to transmission electron microscopy of from 6 to 7 nm, such as 6.6 nm; and/or
the quantum dot has an average hydrodynamic size of from 8 to 9 nm, such as 8.6 nm; and
   (d) the atomic percentages in the quantum dot are as follows: In from 35 to 45%; As from 1 to 5%; P from 25 to 35%; Zn from 5 to 10%; Ga from 5 to 9%; and S from 8 to 15%, optionally wherein the atomic percentages in the quantum dot are as follows: In from 39.7 to 39.8%; As from 2.2 to 2.3%; P from 31.3 to 31.4%; Zn from 8.5 to 8.6%; Ga from 7.5 to 7.6%; and S from 10.3 to 10.4%.

### DRAWINGS

FIG. 1 depicts (a) Schematic representation of the laser-scanning imaging platform for deep fluorescence bioimaging; (b) Schematic of an In(Zn)As—In(Zn)P—GaP—ZnS quantum dot (QD) with its respective bulk semiconductor bandgaps; (c) Absorbance and photoluminescence (PL) spectra; (d) Time-resolved photoluminescence (TRPL) decay; (e) Transmission electron microscopy (TEM) image; and (f) Elemental composition as determined by energy dispersive X-ray spectroscopy (EDX) for In(Zn)As—In(Zn)P—GaP—ZnS QDs.

FIG. 2 depicts the size distribution of the synthesized In(Zn)As—In(Zn)P—GaP—ZnS QDs from the obtained TEM image.

FIG. 3 depicts the schematic for the QDs-resin fluorescent glass panel fabrication.

FIG. 4 shows (a) Photograph (above) and fluorescence image (below) of fluorescent In(Zn)As—In(Zn)P—GaP—ZnS QD glass panel; and (b) Two sets of masks (above) containing vertical bands and the letters “NUS” with their corresponding fluorescence images (below).

FIG. 5 shows (a) From left to right-side view and top view images of real pork loin tissues of 2 mm thickness each that are incrementally stacked over the mask patterns from 2 mm to 16 mm, and the corresponding “Original” and “Processed” fluorescence images. The “Original” images were produced using our laser-scanning imaging platform and the corresponding “Processed” images are obtained after processing of the “Original” images by an artificial neural network; and (b) From left to right-side view of a pork skin tissue of 13 mm thickness, the corresponding top view, and the corresponding “Original” and “Processed” fluorescence images using the two mask patterns.

FIG. 6 shows from left to right-side view and top view images of real pork loin tissues of 2 mm thickness each that are incrementally stacked over the mask patterns from 2 mm to 16 mm, and the corresponding fluorescence images. The images were taken using a Canon 200D DSLR camera that is modified with a 720 nm longpass filter, and with 634 nm red LEDs as a blanket-illumination excitation source.

FIG. 7 depicts the (a) Schematic of the artificial neural network as the machine learning approach to enhance the fluorescence imaging contrast and resolution; (b) Schematic representation of pixel clusters used as the input from the “Original” image being processed into a single pixel output in the “Processed” image; and (c) Resolution against tissue thickness obtained through visual analysis of the “Original” and “Processed” fluorescence images of the pork loin tissue stacked above the vertical-band mask.

FIG. 8 shows (a) Top view, side view, and fluorescence images of a rack of pork ribs that is placed on top of fluorescent In(Zn)As—In(Zn)P—GaP—ZnS QD panel; and (b) Top view and fluorescence images of a human palm placed on top of the fluorescent QD panel.

FIG. 9 depicts the (a) Reaction scheme for the phase transfer of NIR In(Zn)As—In(Zn)P—GaP—ZnS QDs from hexane into water by replacement of OA ligands with MSA ligands at the QD surface; (b) QD-OA (left, before ligand exchange) and QD-MSA (right, after ligand exchange) being dispersed in hexane-water mixture, which formed an immiscible layer; (c) Hydrodynamic size distribution of QD-MSA in water by dynamic light scattering (DLS); (d) PL spectra before and after ligand exchange; and (e) Photo-stability study of QD-MSA in water under three hours of 405 nm CW laser photo-excitation (30 mW).

FIG. 10 shows the (a) TEM images of QD-MSA; and the calculated (b) Size distribution.

FIG. 11 depicts the photo-stability study of the QD-MSA solution in water under 405 nm (30 mW) laser photo-excitation for three hours. The PL spectrum before (black) and after three hours (blue) of laser photo-excitation in air for the QD-MSA solution exhibited no spectral shifts with the PL peak and full-width at half maximum (FWHM) invariant at 828 nm (in dotted lines) and 110 nm, respectively.

FIG. 12 shows the a) Fluorescence confocal microscopy images of HeLa cells incubated with (from left to right) none, 0.5 mg mL⁻¹, and 1 mg mL⁻¹QD-MSA (red). HeLa cells are also stained with nuclear dye Hoechst (blue) and mitochondria dye MitoTracker Green (green); and (b) HeLa cell viability after 24-hours incubation with QD-MSA at varying concentrations.

### DESCRIPTION

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

The methods and apparatus disclosed herein may make use of any suitable nanocrystalline material that capable of fluorescing upon excitation from light from a near-infrared light source. Said materials may be quantum dots or, more particularly, giant shell quantum dots. For example, the methods and apparatus disclosed herein may make use of giant shell quantum dots capable of fluorescing upon excitation from light from a near-infrared light source. Said giant shell quantum dots may have the formula:

In(Zn)As—In(Zn)P—GaP—ZnS

wherein:
In(Zn)As is the core of the quantum dot;
In(Zn)P is the giant shell;
GaP represents an interlayer shell between In(Zn)P and ZnS; and
   ZnS represents an outer layer shell of the quantum dot.

Depending on the desired application for the quantum dots, the ZnS outer layer may may further comprise an organic compound that may be hydrophilic or hydrophobic (or may have both). Hydrophobic organic compounds that may be mentioned herein include, but are not limited to oleic acid. Hydrophilic organic compounds include, but are not limited to, mercaptosuccinic acid. When a hydrophilic organic compound (e.g. mercaptosuccinic acid) is present, it may be further functionalised with a biological targeting agent (e.g. the biological targeting agent may be selected from one or more of the group consisting of folic acid and a cancer-specific antibody). As will be appreciated, the presence of targeting agents may make the giant shell quantum dots so functionalised suitable for use in vivo (e.g. for diagnostic purposes on a human or animal or for research purposes, such as determining the location of a tumour in a subject animal).

The giant shell quantum dots may display any suitable emission peak. For example, the giant shell quantum dots may display an emission peak at from 820 to 850 nm, such as from 828 to 837 nm, such as 828 nm or 837 nm. Additionally or alternatively, giant shell quantum dots may display any suitable photoluminescence lifetime, for example, the quantum dots may display a photoluminescence lifetime of from 20 to 100 ns, such as from 30 to 70 ns, such as from 40 to 60 ns, such as 59 ns. Additionally or alternatively, the giant shell quantum dots may absorb light at any suitable wavelength (i.e. in the near-IR range), for example, the giant shell quantum dots may absorb light at a wavelength of from 400 to 800 nm. Additionally or alternatively, the giant shell quantum dots may display any suitable photoluminescence quantum efficiency. For example, the giant shell quantum dots may display a photoluminescence quantum efficiency of from 60 to 75%.

The giant shell quantum dots may have any suitable size. For example, the quantum dots may have:
an average size according to transmission electron microscopy of from 6 to 7 nm, such as 6.6 nm; and/or
the quantum dot may have an average hydrodynamic size of from 8 to 9 nm, such as 8.6 nm.

The atomic percentages in the giant shell quantum dots may be as follows: In from 35 to 45%; As from 1 to 5%; P from 25 to 35%; Zn from 5 to 10%; Ga from 5 to 9%; and S from 8 to 15%. More particularly, the atomic percentages in the giant shell quantum dots may be as follows: In from 39.7 to 39.8%; As from 2.2 to 2.3%; P from 31.3 to 31.4%; Zn from 8.5 to 8.6%; Ga from 7.5 to 7.6%; and S from 10.3 to 10.4%.

The giant shell quantum dots described herein may be used in any of the applications discussed hereinbelow.

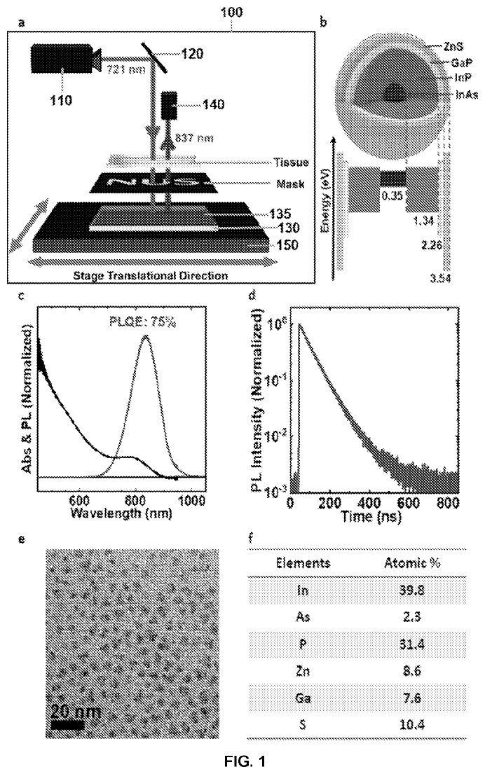

In an aspect of the invention (see FIG. 1a), there is disclosed an imaging device 100 comprising:
a near-infrared light source 110;
a light directing means or apparatus 120;
an array 130 comprising nanocrystals (e.g. giant shell quantum dots) 135 that are capable of fluorescing upon excitation from light from the near-infrared light source; and
a detecting means or apparatus 140 configured to detect light emitted by nanocrystals.

While not necessary, the array 130 may be positioned on a moveable platform 150 such that the array is movable relative to the near-infrared light source and/or a light beam from near-infrared light source is moveable relative to the array.

As will be appreciated, any suitable means of moving the light beam may be used and are well-known. For example, the light source apparatus may be on a moveable apparatus, or the light beam itself may be manipulated using standard techniques using optics.

When used herein, the term “array” simply refers to an arrangement of nanocrystals that can provide the desired effect. For example, the array may be presented as a panel of nanocrystals, such as a panel of giant shell quantum dots.

Any suitable nanocrystals capable of generating the effect listed above may be used, such as quantum dots. More particularly, any suitable giant shell quantum dots capable of generating the effect listed above may be used. Examples of such quantum dots are disclosed hereinbefore.

Any suitable near-infrared light source may be used in this device. For example, the near-infrared light source may be a laser capable of emitting light at near-infrared wavelengths (e.g. 721 nm). Any suitable light directing means or apparatus may be used, for example, the light directing means or apparatus comprises a mirror. Other materials that could be used include optical fibers and the like, as well as combinations.

As will be appreciated, the detecting means or apparatus may further comprise an imaging apparatus. More particularly, the detecting means or apparatus may further comprise an imaging processing unit. The imaging apparatus and/or imaging processing unit may be used to provide an image. This image may be enhanced by the use of the neural network disclosed herein.

As depicted in FIG. 1a. the imaging device may be operated by placing an object to be imaged between the light path from the light source to the array comprising the nanocrystals (e.g. giant shell quantum dots), such that the light passes through the object to be imaged, as does the light fluoresced from the nanocrystals. As will be appreciated, the light generated from the light source may cover a small area and so the object may need to be moved to enable the entire area to be “scanned”. This may be achieved through the use of a translational stage or other apparatus capable of moving the object and/or the light source. This may be in a pre-determined or random pattern (e.g. as required by the imaging apparatus etc.).

Thus, in a further aspect of the invention, there is disclosed a method of imaging a part or the whole of a human or animal body, using an imaging device, comprising:
a near-infrared light source;
a light directing means or apparatus;
a array comprising nanocrystals (e.g. giant shell quantum dots) capable of fluorescing upon excitation from light from the near-infrared light source; and
a detecting means or apparatus configured to detect light emitted by the nanocrystals, where the method comprises the steps of:
   (a) positioning a first side of the part or whole of the human or animal body to be imaged to face the near-infrared light source, light directing means or apparatus and a detecting means or apparatus and a second side of the part or whole of the human or animal body to be imaged to face a array comprising nanocrystals;
   (b) directing near-infrared light from the near-infrared light source through the first and second surfaces of the part or whole of the human or animal body to be imaged via the light directing means or apparatus and into the array comprising nanocrystals; and
   (c) detecting fluorescent light released from the nanocrystals using the detecting means or apparatus.

The method disclosed above may further comprise the step of capturing an image of the part or whole of the human or animal body to be imaged based on the detected fluorescent light, the image being a scattered image. The method may further comprise the step of processing the scattered image using the computerized method described herein to enhance the scattered image.

As will be appreciated, the device used in this method may be the imaging method described hereinbefore.

As noted before, the nanocrystals (and more particularly the quantum dots) disclosed herein may also be used in in vivo applications. Thus, there is also disclosed a method of diagnosis comprising the steps of:

(a) supplying a plurality of nanocrystals (e.g. giant shell quantum dots) capable of fluorescing upon excitation from light from the near-infrared light source to a subject;
(b) subjecting a target site on the subject to light irradiation; and
(c) detecting a signal, or lack thereof, generated by the nanocrystals to provide a diagnosis.

Any suitable nanocrystals capable of generating the effect listed above may be used. More particularly, any suitable giant shell quantum dots capable of generating the effect listed above may be used. Examples of such quantum dots are disclosed hereinbefore. More particularly, the quantum dots may be one that are functionalised with a biological targeting agent (e.g. the biological targeting agent may be selected from one or more of the group consisting of folic acid and a cancer-specific antibody).

The method may be one in which there is a further step of capturing an image of the target site based on the detected signal, the image being a scattered image. Additionally or alternatively, the method may further use a step of processing the scattered image using the computerized method described herein to enhance the scattered image for the diagnosis.

Thus, in a further aspect of the invention, there is disclosed a computerized method for processing scattered images obtained by imaging through scattering media. For example, the scattered images are fluorescence images that may be captured as a result of light fluorescing from the quantum dots. The computerized method comprises steps of:
providing a trained neural network model trained with a training dataset of scattered images comprising associated pairs of low-resolution images and high-resolution images, each image comprising a series of separated bands;
receiving an input scattered image by the trained neural network model;
processing the input scattered image using the trained neural network model; and
generating an output image by the trained neural network model in response to said processing of the input scattered image,
wherein the output image has a higher resolution than the input scattered image.

The computerized method further comprises training the neural network model, said training comprising:
(a) extracting, from the training dataset, an associated pair of low-resolution and high-resolution images;
(b) identifying an input pixel from the low-resolution image and a corresponding true output pixel from the high-resolution image;
(c) selecting a cluster of pixels from the low-resolution image, the cluster of pixels surrounding the input pixel;
(d) weighting each pixel in a cluster of pixels using a set of weight and bias parameters;
(e) generating a processed output pixel from said processing of the cluster of pixels;
(f) determining an error between the processed output pixel and the true output pixel;
(g) backpropagating the error to adjust the parameters in step (d);
(h) iteratively performing steps (d) to (g) to minimize the error,
wherein the minimized error is associated with an optimized set of parameters for the neural network model.

It will be appreciated that the scattering media may be any suitable scattering media. For example, it may be the flesh covering the bones of a human or animal, or it may be a frosted pane of glass, obscuring an object behind it.

Further aspects and embodiments of the invention will now be described by reference to the following non-limiting examples. As will be appreciated, the exact methods used for each of the above aspects and embodiments may be derived directly or by analogy from the following examples.

### EXAMPLES

Materials

Indium acetate (99.99%), zinc acetate (99.99%), gallium (III) chloride (99.99%), sulfur (99.5%), trioctylphosphine (TOP, 97%), zinc acetate dihydrate (99%), ammonium hydroxide solution (28-30% v/v in water), mercaptosuccinic acid (MSA, 99%), tri-p-tolyl phosphine (98%), tricyclo[5.2.1.02,6]decanedimethanol diacrylate (TCDDA), paraformaldehyde (PFA, reagent grade) and 2,2-bis(hydroxymethyl)propionic acid (DMPA, 98%) were purchased from Sigma-Aldrich, and used without further purification. 1-octadecene (ODE, 90%) was purchased from Sigma-Aldrich, and dried with activated molecular sieves in a round-bottom flask (RBF) and degassed under vacuum for 30 minutes before use. Both octylamine (99%) and oleic acid (OA, 90%) were purchased from Sigma-Aldrich, and degassed under vacuum before use. 3-(4,5-Thiazolyl Blue tetrazolium bromide (MTT) was purchased from Alfa Aesar. Tris(trimethylsilyl)phosphine (TMS3P, 10% v/v in hexane) was purchased from Alfa Aesar, and concentrated by the removal of hexane under reduced pressure. Tris(trimethylsilyl)arsine (TMS3As) was synthesized according to literature procedures (Wells, R. L. et al., Inorg. Synth. 1997, 31, 150). Tris(trimethylsilyl) arsine, tris(trimethylsilyl)phosphine and gallium (III) chloride are pyrophoric and must be handled carefully in a moisture-free and oxygen-free environment. Isobornyl acrylate (IBOA, technical grade) was purchased from Sigma-Aldrich, and pre-polymerised by mixing IBOA with 0.25 wt % DMPA. The resulting mixture was degassed for 10 minutes and then photo-polymerised with UV lamp (365 nm, 46 W) for 30 seconds. Dimethylsulfoxide (DMSO, 99.9% analytical grade), Hexane (98.5% high performance liquid chromatography, or HPLC grade) and MitoTracker™ Green FM were purchased from Thermo Fisher Scientific and used without further purification. Chloroform (99.8% analytical grade) and ethanol absolute (99.8% analytical grade) were purchased from VWR Chemicals BDH® and used without further purification. Roswell Park Memorial Institute (RPMI) 1640 Medium was purchased from Cytiva and used without further purification. Phosphate buffered saline (PBS, ultra-pure grade) was purchased from Vivantis and used without further purification. Hoechst 33342 trihydrochloride trihydrate (10 mg/mL solution in water) was from Life Technologies Corporation and used without further purification.

Analytical Techniques

UV-Visible Absorbance Measurements

UV-visible absorbance spectra were obtained by measuring the transmitted light intensity of an Ocean Optics HL-2000 broadband light source, using an Ocean Optics Flame-T and Flame-NIR spectrometer.

Photoluminescence Quantum Efficiency (PLQE) Measurements

The PL spectra and PLQE were obtained by photo-exciting the samples in an integrating sphere, using a Spectra-Physics 405 nm (100 mW, CW) diode laser, and measuring the absorption and PL using a calibrated Ocean Optics Flame-T and Flame-NIR spectrometer.

TEM and EDX

TEM images were recorded using JEOL JEM-2100F Field Emission TEM operated at 200 kV. This system was equipped with an Oxford Instruments INCA EDX. TEM samples were prepared by diluting QD-OA solutions in hexane and QD-MSA solutions in water, then drop-casting the solution on a copper grid.

Time-Resolved Photoluminescence (TRPL)

TRPL decays were acquired using a time-correlated single photon counting (TCSPC) setup (Horiba FluoroLog-3 Spectrofluorometer). Samples were excited using a 438 nm nano-LED light source (Horiba NanoLed-440L) with a typical pulse width of 260 ps and a repetition rate of 500 kHz. The PL decay curves were fitted using an exponential equation shown in equation (1) where A are the amplitudes of the exponential terms while r is the PL lifetime. I is the normalized PL intensity and t is the time.

$$I ( t ) = A exp ( - \frac{t}{τ} ) \qquad ( 1 )$$

The PLQE is defined as the ratio of the radiative recombination rate constant (Γᵣ) to the sum of the radiative and non-radiative recombination rate constant (Γₙᵣ), given by equation (2):

$$PLQE = \frac{{Γ}_{r}}{{Γ}_{r} + {Γ}_{nr}} \qquad ( 2 )$$

The PL lifetime can be expressed as the reciprocal of the sum of recombination rate constants:

$$τ = \frac{1}{{Γ}_{r} + {Γ}_{nr}} \qquad ( 3 )$$

Therefore, using equation (2) and (3), we can calculate the value of Γᵣ and Γₙᵣ:

$${Γ}_{r} = \frac{PLQE}{τ} \qquad ( 4 )$$

$${Γ}_{nr} = \frac{1 - PLQE}{τ} \qquad ( 5 )$$

### Example 1. Laser-Scanning Imaging Platform

In an ideal scenario, the laser beam penetrates the tissue with minimal scattering and attenuation, and excites the fluorescent QDs. The fluorescence signal would then traverse across the same tissue thickness to reach the detector and form the image. Therefore, we have designed our laser source and our QDs to both function within the near-infrared (NIR) spectral region to take advantage of the fact that NIR light experiences significantly lower attenuation and scattering by biological tissues as compared to visible photons (Weissleder, R., Nat. Biotechnol. 2001, 19, 316-317; and Frangioni, J. V., Curr. Opin. Chem. Biol. 2003, 7, 626-634). Here, we reported a laser-scanning imaging platform that employed a NIR laser and a high-efficiency NIR QD fluorescent dye to perform deep imaging of larger biological systems.

As shown in FIG. 1a, our in-house-built imaging setup comprised a two-axis motorized translation stage with an area coverage of 300×300 mm². A stationary laser beam with a wavelength of 721 nm and beam diameter of <1 mm was directed perpendicularly to the top of the translating stage. A photomultiplier tube (PMT), with bandpass filters accepting photons within 825 nm and 875 nm range, was affixed above the stage to detect fluorescence signal that was excited through the laser source. The stage, which held the fluorescent sample, was programmed to translate in a rastering style such that the laser beam scans through the entire stage area. The row-by-row signal readout from the PMT, using a digital multimeter, then formed the full fluorescence image. We employed a laser-scanning approach for our imaging setup due to its significantly smaller excitation volume as compared to conventional wide-field fluorescence imaging techniques, which directly translated to a markedly-improved imaging contrast and resolution due to the reduction of undesired fluorescence signals from areas outside the point of laser excitation. This principle is similar to laser-scanning concepts used in confocal microscopy systems, but deployed on a larger scale. In a typical imaging experiment, we utilized a fluorescent array in the form of a panel that was loaded with 837 nm NIR-emitting QDs, and applied a black mask above to provide customized fluorescent patterns.

We designed two sets of black masks that were placed above the fluorescent glass panel to provide customized fluorescent patterns for imaging. One pattern comprised spatially separated vertical bands, and another comprised the letters “NUS.”

### Example 2. Synthesis of In(Zn)As—In(Zn)P—GaP—ZnS QDs

We prepared our NIR QDs using a convenient one-pot continuous injection methodology (Franke, D. et al., Nat. Commun. 2016, 7, 12749; Wijaya, H. et al., Chem. Mater. 2019, 31, 2019-2026; and Wijaya, H. et al., Adv. Funct. Mater. 2020, 30, 1906483).

In(Zn)Oleate Solution

Indium acetate (0.25 mmol, 73 mg), zinc acetate (0.25 mmol, 46 mg) and OA (1.875 mmol, 0.67 mL) were mixed with ODE (8.5 mL) at room temperature (RT). Vacuum was applied to the round-bottom flask (RBF) and the reaction mixture was heated to 80° C. for 30 minutes under vacuum. The reaction mixture was then purged with argon and heated to 160° C., and stirred for 1 hour to form a clear solution. The mixture was subsequently cooled to 80° C. and vacuumed for 30 minutes before back-filling the RBF with argon and cooling the In(Zn)oleate solution to RT.

In(Zn)as Precursor

In a 5 mL RBF, In(Zn)oleate solution (0.45 mL, 0.0125 mmol) was mixed at RT with 0.55 mL of arsine precursor solution (made up of 0.55 mL of ODE, 4.6 μL, 0.0125 mmol of TMS3As and 31 μL of octylamine). The resulting In(Zn)As precursor solution was stirred for 5-10 minutes at RT before being used for injection.

In(Zn)P Precursor

In a 50 mL 2-neck RBF, In(Zn)oleate solution (8.5 mL, 0.25 mmol) was mixed at RT with 1 mL of phosphine precursor (made up of 73 μL, 0.25 mmol of TMS3P, 0.5 mL of octylamine and 0.5 mL of ODE). The resulting In(Zn)P precursor solution was stirred for 10 minutes at RT before being used for injection.

TOP-Sulfur (TOP-S) Precursor

Sulfur (0.50 mmol, 16 mg) was added to ODE (4.5 mL) in a RBF and vacuumed at 80° C. for 30 minutes. The reaction mixture was then filled with argon before addition of TOP (1.125 mmol, 0.52 mL) to form the TOP-S precursors.

Zn(Oleate)₂ Solution

A Zn(oleate)₂ solution was prepared from zinc acetate (0.50 mmol, 91.7 mg), OA (1.125 mmol, 0.4 mL) and ODE (to make 5 mL) based on the protocol for In(Zn)oleate solution except the Zn(oleate)₂ solution needed to be heated to 80° C. to form a clear solution for injection.

In(Zn)as Seed Solution

Indium acetate (0.10 mmol, 30 mg), zinc acetate (0.05 mmol, 10 mg) and OA (0.0375 mmol, 13.2 μL) were mixed at RT with ODE (5 mL) in an argon-filled 50 mL RBF. The reaction mixture was vacuumed and heated to 80° C. for 30 minutes. The reaction mixture was then back-filled with argon, heated to 160° C. and stirred for 1 hour to form a clear and colorless solution. Following that, the reaction mixture was cooled to 80° C. and vacuumed for 30 minutes. The RBF was then filled with argon and heated to 230° C. An arsine precursor solution was prepared by mixing TMS3As (0.066 mmol, 20 μL) and octylamine (0.20 mL) with ODE (to make 1 mL) under an inert argon glovebox environment. The arsine precursor solution was injected into the indium precursor solution at 230° C. over 5 seconds. After stirring at 230° C. for 2.5 hours, a small portion (0.37 mL, 0.005 mmol) of the seed solution was withdrawn and diluted with dry ODE (2.5 mL) in another 3-neck 100 mL RBF. The remaining amount of the In(Zn)As seed solution was stored in the glovebox under argon environment for subsequent use.

InZn(As)—In(Zn)P

The diluted In(Zn)As seed solution was vacuumed at 80° C. for 15 mins before purging the reaction mixture with argon and heating to 230° C. The In(Zn)As precursor solution (1 mL) was injected into the In(Zn)As seed solution at 230° C., using a syringe pump, at a rate of 0.1 mL/min over 10 minutes. The solution was stirred for another 1 hour to give a In(Zn)As core that emits at ˜710 nm. The In(Zn)P precursor solution was injected into the In(Zn)As reaction mixture at 230° C., using a syringe pump, at a rate of 0.1 mL/min. The temperature was raised to 240° C. after 33 minutes, and to 250° C. after 66 minutes. After complete injection at 100 minutes, the temperature was raised to 260° C. and the solution was stirred for another 30 minutes to expend all precursors and complete the In(Zn)P shell synthesis. The slow injection process kept the concentration of the precursors low in the reaction mixture and significantly suppressed undesired side nucleation of In(Zn)P, while promoting the continuous growth of the thick shell. The reaction mixture was cooled to 240° C. for 7 minutes before the end of the In(Zn)P shell synthesis stage. Aliquots of the reaction were withdrawn from the reaction mixture and diluted in anhydrous hexane to track the reaction progress by optical absorbance and PL measurements.

In(Zn)As—In(Zn) P—Gap

Gallium (III) chloride (0.125 mmol, 22 mg) and OA (1.875 mmol, 0.146 mL) were mixed with ODE (to make 3.75 mL) in an argon-filled RBF under an inert argon glovebox environment to give a Ga(oleate)₃ precursor solution. The Ga(oleate)₃ precursor solution was stirred and degassed at RT for 2 hours under vacuum until a clear, pale yellow solution was observed. The Ga(oleate)₃ precursor solution (3.75 mL) was injected into the In(Zn)As—In(Zn)P reaction mixture at 240° C., using a syringe pump, at a rate of 0.15 mL/min. After complete injection at 25 minutes, the reaction mixture was stirred for another 25 minutes at 240° C. to expend all precursors and complete the GaP shell synthesis. Gallium substituted the surface indium on In(Zn)As—In(Zn)P to give a GaP interlayer (Kim, S. et al., J. Am. Chem. Soc. 2012, 134, 3804-3809). This GaP interlayer eases the lattice mismatch between the In(Zn)P and ZnS shells, and offers significant enhancement to the PLQE.

In(Zn)As—In(Zn)P—GaP—ZnS QDs

The TOP-S precursor solution (5 mL) was injected into the In(Zn)As—In(Zn)P—GaP reaction mixture at 250° C., using a syringe pump at a rate of 0.25 mL/min. After complete injection at 25 minutes, the reaction mixture was stirred for another 25 minutes at 250° C. to expend all precursors. This was followed by another injection of the TOP-S precursor solution (5 mL) and Zn(oleate)₂ solution (5 mL) at 260° C., using a syringe pump at a rate of 0.25 mL/min. The reaction mixture was then stirred for another 25 minutes at 260° C. to expend all precursors and complete the ZnS shell layer. The reaction mixture was allowed to cool to RT. Ethanol (40 mL) was added to the reaction mixture to precipitate the In(Zn)As—In(Zn)P—GaP—ZnS QDs, followed by centrifugation of the mixture at 6000 rpm for 5 minutes. The clear supernatant was carefully removed using a dropper. The addition of ethanol and centrifugation process was repeated twice to purify the quantum dots. The final precipitate was re-dispersed in anhydrous hexane (20 mL) and stored for further use.

### Example 3. Characterization of the QDs

The In(Zn)As—In(Zn)P—GaP—ZnS QDs prepared in Example 2 (QD-OA) were characterized.

### Results and Discussion

Our new NIR-emitting In(Zn)As—In(Zn)P—GaP—ZnS QD (FIG. 1b) had a remarkably high PLQE of 75%. The PL spectrum of the In(Zn)As—In(Zn)P—GaP—ZnS QDs in FIG. 1c showed a peak emission at 837 nm with a FWHM of 110 nm. The QDs absorbed strongly across a broad spectral range from 400 nm to 800 nm, hence allowing a versatile selection of excitation laser source, even into the NIR region. TRPL measurements on the QDs (FIG. 1d and Table 1) revealed a primarily monoexponential PL decay that could be attributed to the radiative recombination process, given the high PLQE of the QDs. Crucially, an ultra-short PL lifetime of 59 ns was observed, which is remarkably valuable for the laser-scanning fluorescence imaging approach. The short PL lifetime indicates that the imaging speed is limited only by the laser-scanning and the data acquisition speeds, both of which are easily fulfilled by modern galvo-mirror systems and data acquisition instruments, respectively. This is in contrast to dopant-based NIR phosphors or lanthanide upconversion emitters that possess long emission lifetimes exceeding hundreds of microseconds (Lu, Y. et al., Nat. Photonics 2013, 8, 32; and Chen, G. et al., ACS Nano 2012, 6, 8280-8287).

TABLE 1
Table summarizing the calculated PL lifetime parameters
of QD-OA. The values of Γᵣ and Γₙᵣ were calculated
using equation (5) shown above.

| Sample | PLQE/% | τ/ns | Γᵣ/μs⁻¹ | Γₙᵣ/μs⁻¹ |
|---|---|---|---|---|
| QD-OA | 75.4 | 58.5 | 12.9 | 4.2 |

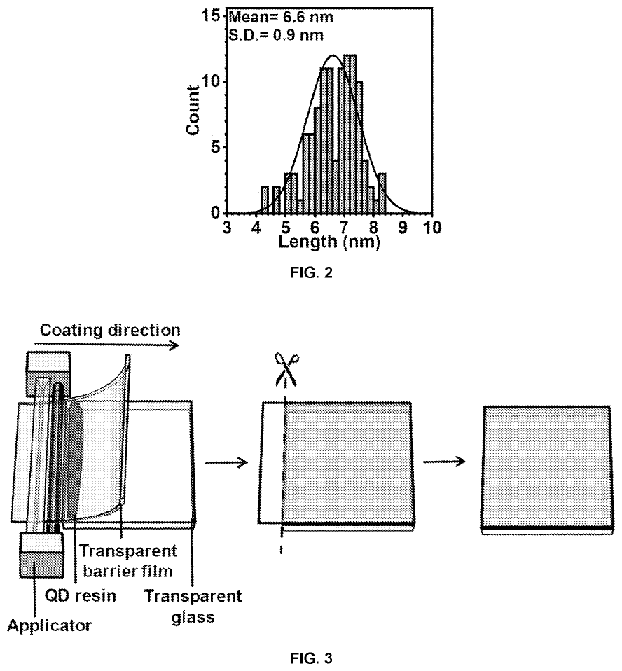

The transmission electron microscopy (TEM) image of the QDs (FIG. 1e) revealed irregularly-shaped QDs with a uniform size distribution and an average size of 6.6 nm (FIG. 2). We measured the elemental composition of the QDs by energy dispersive x-ray spectroscopy (EDX) and tabulated the results in FIG. 1f. Our QDs were shown to possess a low As content of only 2.3 atomic % that was located primarily at the inner core, and confirmed the successful realization of a giant-shell design using our synthetic approach.

### Example 4. QDs-Resin Fluorescent Glass Panel Fabrication

To test the imaging functions of our setup in Example 1, we prepared a fluorescent glass panel by casting a dispersion of NIR-emitting QDs (prepared in Example 2) in a UV-curable resin onto a clear borosilicate glass plate, followed by photocuring the QD-resin under a 365 nm UV illumination.

The QD-resin comprises a homogenous dispersion of QDs in IBOA monomers and TCDDA crosslinkers. To synthesize the QD-resin, 15 mL of the QDs solution was centrifuged in ethanol at 10,000 rpm for 5 minutes. The supernatant was removed and the solid was re-dispersed in chloroform (5 mL). Tri-p-tolyl phosphine (600 μL, 100 mg/mL in chloroform) was added to the solution and stirred for 30 minutes. The solvent was vacuum-evaporated and the resulting solid was re-dispersed in IBOA (500 μL). Pre-polymerised IBOA (5 mL, viscosity ˜1700 cP) and TCDDA (2.25 mL) were added sequentially and the reaction mixture was stirred for 10 minutes between each addition. Finally, a radical photoinitiator, DMPA (37.5 mg, 0.5 wt %), was added and the reaction mixture was stirred for 3 hours to form a clear and homogenous QD-resin mixture (viscosity ˜1100 cP). The resulting resin mixture was then applied onto a transparent glass (16×16 cm² with 0.6 mm thickness) using a film applicator to achieve a uniform 200 μm thick film (FIG. 3). The film was finally photo-cured using Spectrolinker™ XL-1500 at 144 000 μJ/cm² for 64 seconds.

### Example 5. Imaging Experiments to Produce Fluorescent Patterns

We used the two designed sets of masks for coupling with the QDs-resin fluorescent panel (prepared in Example 4) to produce fluorescent patterns in the imaging experiments.

### Results and Discussion

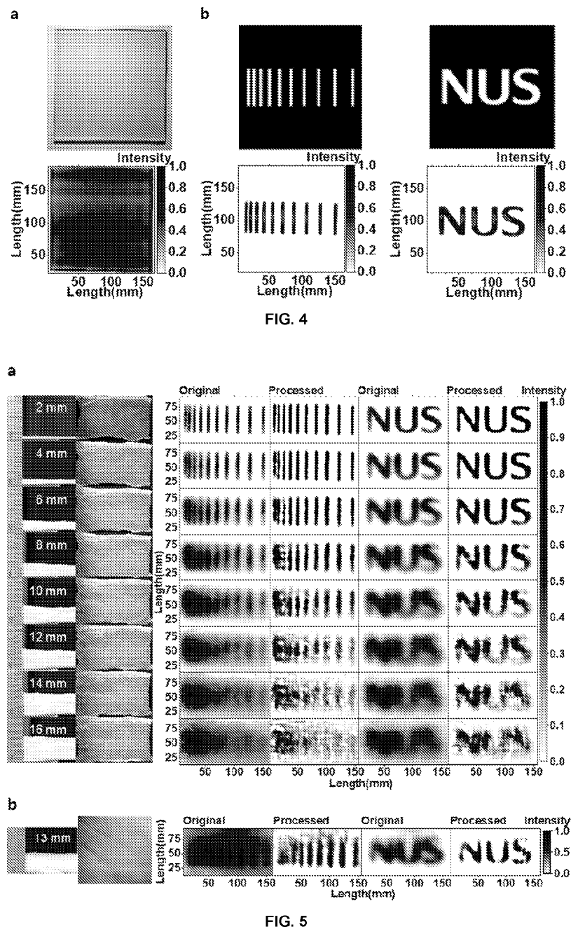

FIG. 4a shows an image of the fluorescent panel under ambient lighting, taken using a Canon EOS M100 camera, and a fluorescence image, acquired using our laser-scanning imaging setup. Slight horizontal bands could be observed on the fluorescent panel as our imaging setup was sufficiently sensitive to pick up minor non-uniformities in the coating by a manual film applicator. Then, the two designed sets of masks were coupled with the fluorescent panel to produce fluorescent patterns. The mask patterns and the corresponding fluorescence images are shown in FIG. 4b. The vertical-band mask was designed with increasing spacings of 2, 4, 6 . . . , 18 mm for the purpose of determining the imaging resolutions at varying tissue depths.

### Example 6. Real Pork Loin Tissues for Imaging Experiments

To better mimic the compositional complexity of actual animal tissues, we employed real pork loin tissues for our imaging experiments instead of the more commonly-used synthetic tissue phantom. Layers of pork loin tissues with controlled thicknesses were placed successively above the fluorescent patterns to simulate fluorescence in a deep-tissue environment, and images were acquired using our laser-scanning imaging setup. The pork loin tissue was thinly-sliced to give 2 mm layers and stacked incrementally over the mask patterns for up to a total of 8 layers (16 mm) as shown in FIG. 5a.

### Results and Discussion

FIG. 5a shows the imaging results of fluorescent patterns that were overlaid with thin slices of pork loin tissues. As expected, the mask pattern was barely noticeable under visible-light camera imaging with an overlaid tissue thickness of only 2 mm, and completely nondistinguishable beyond that. In comparison, NIR fluorescence images (in columns labelled ‘Original’) that were acquired through our laser-scanning setup showed distinct features of the mask patterns at tissue thicknesses beyond 10 mm. Despite the fluorescence images becoming fuzzier due to increased scattering at greater tissue depths, broad features of the vertical bands and the ‘NUS’ letters could still be identified up to 16 mm. We repeated the imaging experiment using a 13 mm pork skin (FIG. 5b) to simulate non-invasive in vivo animal studies, where photoexcitation is performed through skin, and observed improved imaging results at similar tissue thicknesses. This is likely due to the more homogeneous composition of the pork skin tissue, hence resulting in reduced light scattering.

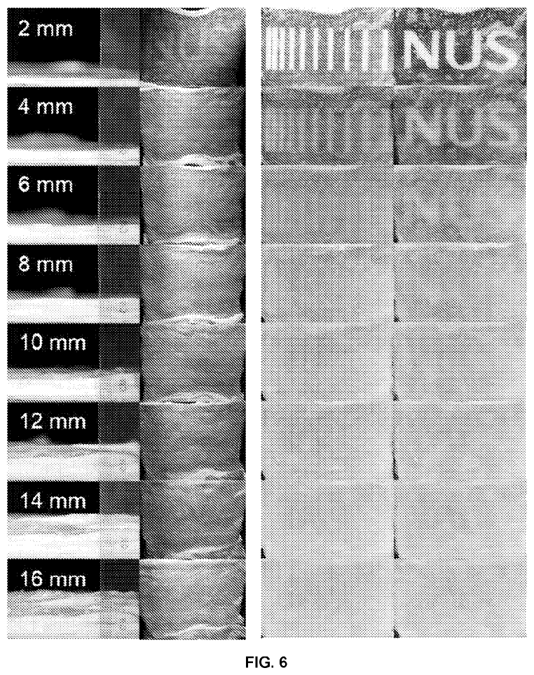

For the purpose of comparison, we also performed wide-field of view (FOV) fluorescence imaging using a Canon 200D DSLR camera that was modified with a 720 nm longpass filter, and used 634 nm red LEDs as a blanket-illumination excitation source. As shown in FIG. 6, the fluorescent patterns were only distinguishable for up to 6 mm of tissue thickness. The fluorescence signal was quickly overwhelmed by the light scattered from the pork tissues at larger thicknesses. This control experiment has thus shown that our laser scanning approach could indeed provide a significantly improved imaging depth compared to conventional imaging methods.

### Example 7. Machine Learning Approach to Enhance Fluorescence Imaging Results

We sought to further enhance our fluorescence imaging results through the processing of the images using a new machine learning approach. The broad idea involved supplying the machine learning algorithms with scattered images and sharp images, and training the machine to process future scattered images into sharper images.

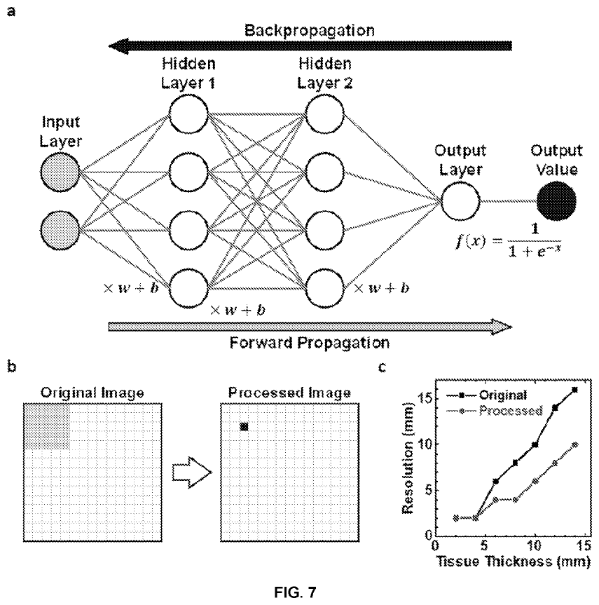

In some embodiments, the scattered images (such as fluorescence images) are processed using a trained machine learning model such as a neural network model. FIG. 7a illustrates an example of the neural network model for processing the scattered images. The neural network model is operative on a computing device that contains one or more processors. The computing device may include a personal computer, laptop, server, mobile device, etc. The neural network model is not limited to any software platform or programming language, and the neural network model may be executed using any number of known platforms and/or languages.

There is a computer-implemented or computerized method for processing scattered images using the neural network model that has been trained beforehand. The method comprises a step of providing the trained neural network model trained with a training dataset of scattered images comprising associated pairs of low-resolution images and high-resolution images, each image comprising a series of spatially separated bands. For each associated pair of images, there is a low-resolution image and a high-resolution image of the same sample or region of interest. The low-resolution images are scattered or blurry images, and the high-resolution images have sharp contrast. The high-resolution images are better than the low-resolution images at least in terms of one or more of spatial resolution, contrast, sharpness, and signal-to-noise ratio.

The neural network model comprises a plurality of layers including an input layer, zero or more hidden layers, and an output layer, each layer having a respective set of neurons for processing data. The input layer is configured for receiving input data from an external source, performing calculations via its neurons, and sending the results onto the subsequent layers. The output layer is configured for receiving inputs from the preceding layers (including the processed input data at the input layer), performing calculations via its neurons, and generating the final output data. Optionally, the neural network model comprises at least one hidden layer residing between the input and output layers. As shown in FIG. 7a, the neural network model has two hidden layers. The first hidden layer processes the input data using a respective set of weight and bias parameters and outputs the results to the second hidden layer. The second hidden layer similarly processes using a respective set of weight and bias parameters and outputs to the output layer. The output layer receives the inputs from the second hidden layer and generates the final output data.

The method comprises steps of receiving (at the input layer) an input scattered image by the trained neural network model, processing (at the hidden layers) the input scattered image using the trained neural network model, and generating (at the output layer) an output image by the trained neural network model in response to said processing of the input scattered image. The output image has a higher resolution than the input scattered image. Training of the neural network model with the training dataset of low-resolution images and high-resolution images enables the neural network model to process subsequent low-resolution images into high-resolution images. For example, the input image has low resolution (e.g. scattered/blurry) and the trained neural network model enhances the image into a high-resolution one by de-scattering the image and improving the contrast and sharpness. The output image may be referred to as a de-scattered image.

The neural network model is initially trained by the training dataset of scattered images which can be acquired by various methods. For example, the scattered images are fluorescence images which are acquired by the near-infrared laser-scanning excitation and efficient quantum dot photoluminescence methods described herein. The neural network model may be pretrained or the method may comprise training the neural network model.

With reference to FIG. 7b, the training comprises a step (a) of extracting, from the training dataset, an associated pair of low-resolution and high-resolution images, a step (b) of identifying an input pixel from the low-resolution image and a corresponding true output pixel from the high-resolution image, and a step (c) of selecting a cluster of pixels from the low-resolution image, the cluster of pixels surrounding the input pixel. The input layer is selected from the cluster of pixels from the low-resolution image.

The training further comprises a step (d) of weighting each pixel in a cluster of pixels with a set of weight and bias parameters. Specifically, each layer multiplies the value of each pixel by the respective weight parameter and adds the respective bias parameter, and sends the resultant values to the next layer. In some embodiments, the step (d) comprises successively weighting each pixel at least twice. For example as shown in FIG. 7a, the pixels are weighted three times—once at the input layer and twice at the two hidden layers.

The training further comprises a step (e) of generating a processed output pixel from said weighting of the cluster of pixels. Notably, as shown in FIG. 7b, a single output pixel is generated from the processing of the cluster of pixels surrounding the input pixel corresponding to the output pixel. The reason for this is that, for fluorescence images, the fluorescence intensity at any one point (pixel) will spread over an area (cluster of pixels) during light scattering, and hence may be back-derived by learning from a sufficiently large library of scattered images. The step (e) may comprise generating (at the output layer) a raw output pixel from said weighting of the cluster of pixels, and generating the processed output pixel (as the final output value) from the raw output pixel using a logistic function. Each final output value represents a single pixel on the processed image.

The training further comprises a step (f) of determining an error between the processed output pixel and the true output pixel from the corresponding high-resolution image. The error may be calculated as a loss function using a supervised batch learning approach, and the error may comprise a mean squared error between the value of the processed output pixel and the value of the true output pixel.

The training further comprises a step (g) of backpropagating the error to adjust the parameters in step (d) using suitable backpropagation algorithms. With an adjusted set of weight and bias parameters, the pixels from the low-resolution image are processed again to thereby regenerate the processed output pixel. The training further comprises a step (h) of iteratively performing steps (d) to (g) to minimize the error, thus improving the accuracy of the processed output pixel. For example, the training comprises using a gradient descent function as a to adjust the parameters and minimize the error (loss function). The gradient descent function is an optimization algorithm used to determine the parameters that minimize a cost function of the training images. The gradient descent function involves multiple iterations of forward propagating the inputs, backpropagating the error, and adjusting the parameters until an optimized set of parameters is determined. The parameters may first be randomly selected and then iteratively processed until they are optimized.

Therefore, the iterative process minimizes the error, and the minimized error is associated with an optimized set of parameters for the neural network model. Further, the training comprises repeating steps (a) to (h) for each associated pair of images in the training dataset, thus completing the training of the neural network model. The optimized weights and bias parameters can then be used to process and enhance subsequent low-resolution images into high-resolution images.

### Results and Discussion

The trained neural network model was tested using two sets of low-resolution scattered images (fluorescence images) as shown in FIG. 6. The low-resolution input images are labelled under “Original” and the processed output images are labelled under “Processed”. The first set of images has spatially separated bands (shown as vertical bands) and the second set of images has the letters “NUS”. The images were created using two black masks (as shown in FIG. 4b) placed on the fluorescent glass panel to provide customized fluorescent patterns for imaging. The first black mask has spatially separated vertical bands and the second black mask has the letters “NUS”. Additionally, the first mask has the vertical bands designed with increasing gaps of 2, 4, 6, . . . , 18 mm or the purpose of determining the imaging resolutions at varying tissue imaging depths. The results of the image processing indicate that the trained neural network model was very successful in improving the overall quality and sharpness of the low-resolution images, even in more complex patterns such as the letters “NUS.”

Moreover, for the first set of images, in order to better quantify the resolution of the images, the gap distances at which two separate bands could no longer be distinguished from one another were analysed. As shown in FIG. 7c, the gap distances were higher for the original images than for the processed images. This shows that the processed images have an improved and finer resolution than the original images. Notably, the resolution improved by approximately twofold.

As mentioned above, each image in the training dataset used to train the neural network model comprises a series of spatially separated bands. The bands can be oriented in any direction, such as vertically. The spatially separated bands in the low-resolution and high-resolution images for training help the neural network model to identify and resolve features at varying gap distances. This improves the ability of the trained neural network model to process and enhance low-resolution images, as evidenced in the results above. Even though the training was performed with images having the spatially separated bands, the trained neural network model was equally capable of resolving features on the “NUS” letters. This generality allows the trained neural network model to be employed to enhance a broad range of scattered images including fluorescence images where scattering problems are observed.

By implementing a trained neural network model, the imaging resolution can be enhanced, paving a promising way forward for the use of machine learning to enhance imaging quality in highly scattering media. As an example, the trained neural network model can be used to process a low-quality image of an item hidden behind a scattering medium such as a piece of frosted glass. The trained neural network model is able to enhance the light information coming through the scattering medium and improve the quality and resolution of the image of the item. The image enhancement through the trained neural network model may also have a far-reaching impact on how future images with significant blurring could be effectively repaired. The trained neural network model can be combined with the laser-scanning approach for fluorescence imaging to develop a low-cost yet powerful methodology for performing non-invasive deep-imaging in larger animal models or human tissues.

### Example 8. Imaging of an Animal Body and Adult-Human Palm

We reasoned that the ability to resolve millimetre-scale features at tissue depths greater than 10 mm was sufficient for performing imaging functions across sections of an animal body or the human body, for instance, through the palm.

In a new experiment, we separately placed a rack of pork ribs (20 mm thickness) and rested an adult-human palm on a fluorescent QD panel, and performed laser-scanning imaging with our setup. We noted that a mild 200 mW/mm² CW laser irradiance at a non-ionising NIR wavelength of 721 nm causes no damage to the skin or tissue, especially when rapidly scanned across the objects.

### Results and Discussion

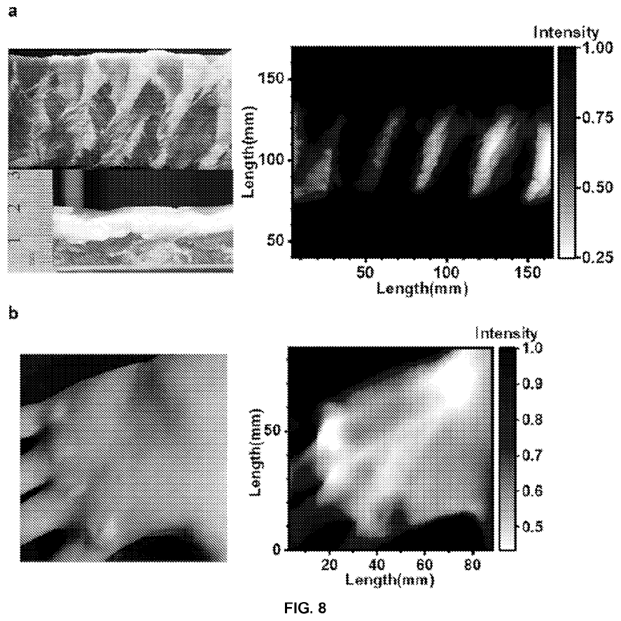

FIGS. 8a and 8b show the fluorescence images of the pork ribs and human palm, respectively, acquired above the fluorescent QD panel. In both sets of the images, the light-colored regions show rather distinct features of the bones that are deep within the tissues. We deduced that the imaging of bone structure was possible since the denser bones attenuate the laser excitation more significantly than the soft tissues, hence blocking out the laser excitation of fluorescence in the QD panel. This surprising discovery marked an important step towards the use of mild NIR radiation to image bone structures in human body and could potentially be applied for clinical pre-X-ray screening in patients, for instance, in paediatric care.

Through the use of laser-scanning excitation and the deployment of artificial neural network for image processing, we have shown that deep imaging performance could be easily achieved across thick sections of animal tissues over a wide FOV. We anticipate that our approach could offer notable improvements to existing in vivo fluorescence imaging and tomography techniques, and could serve as a powerful new research platform for preclinical immunotherapy or targeted therapy studies.

### Example 9. MSA-Capped QDs (QD-MSA) Solution

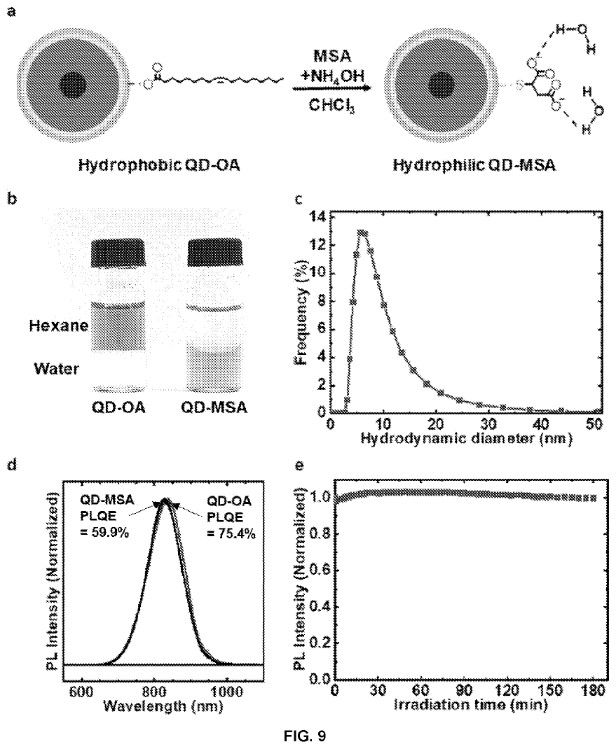

Prior to testing the cytotoxicity of the QDs on HeLa cells, a ligand-exchange step was conducted to replace hydrophobic OA ligands with hydrophilic MSA ligands via a phase transfer method (FIG. 9a) (Yong, K.-T. et al., ACS Nano 2009, 3, 502-510; and Bharali, D. J. et al., J. Am. Chem. Soc. 2005, 127, 11364-11371).

2 mL of the In(Zn)As—In(Zn)P—GaP—ZnS QDs dispersed in hexane (prepared in Example 2) was dried using a rotary evaporator and re-dispersed in chloroform (2 mL). In a 20 mL glass vial, MSA (300 mg, 2 mmol) was stirred in chloroform (5 mL) for 15 minutes until a suspension was formed. The 2 mL of QDs in chloroform was then quickly added to the MSA suspension, and the reaction mixture was stirred vigorously in air. After 1 minute of stirring, ammonium hydroxide solution (28-30% v/v in water, 0.5 mL) was added, and the stirring was continued for another 3 hours. The QDs were then precipitated with ethanol (10 mL) in a 15 mL centrifuge tube, and centrifuged at 10,000 rpm for 5 minutes. The supernatant was discarded, and the residue was dispersed in deionized water (1.5 mL) before being precipitated in ethanol (10.5 mL) and centrifuged at 10,000 rpm for 5 minutes. The purification process involving ethanol addition, centrifugation and water dispersion was repeated twice. After the third centrifugation, the residue was dried under vacuum for 1.5 hours and dispersed in deionized water to obtain a mass concentration of 30 mg/mL. The concentrated QD-MSA solution was filtered through a 0.45 μm hydrophilic syringe filter into a clean 2 mL vial and stored in the freezer for subsequent use and characterization.

### Example 10. Characterization and Stability of QD-MSA

The QD-MSA prepared in Example 9 were characterized. The stability of QD-MSA was also evaluated to validate the applicability of our NIR QDs for bioimaging applications.

Dynamic Light Scattering (DLS) Measurements

The hydrodynamic size distribution (by volume) and zeta (ζ) potential of the MSA-capped QDs in water was estimated using a dynamic light scattering molecular size analyser (Zetasizer Nano ZS using a He—Ne 633 nm laser). The MSA-capped QD solution was first diluted in deionized water to a concentration of 1.5 mg mL⁻¹ before it was filtered through a 0.45 μm hydrophilic syringe filter membrane to remove any residual dust particles. After that, the size distribution and ζ potential were measured.

Stability Study

QD-OA is soluble in hexane while QD-MSA is soluble in water. QD-OA (1 mL, prepared in Example 2) was mixed with deionized water (1 mL) in a 2 mL vial while QD-MSA (1 mL, prepared in Example 9) was mixed with hexane (1 mL) in another 2 mL vial. As the solution mixture settled, QD-OA remained in the hexane phase while QD-MSA remained in the water phase.

Photo-Stability Study

QD-MSA (1.5 mg/mL in water in 1 cm path length cuvette, absorption 36%) was subjected to continuous laser irradiation (30 mW, 405 nm) for 3 hours. PL spectra was taken at timed intervals, and the peak intensity at 825 nm was plotted against time.

### Results and Discussion

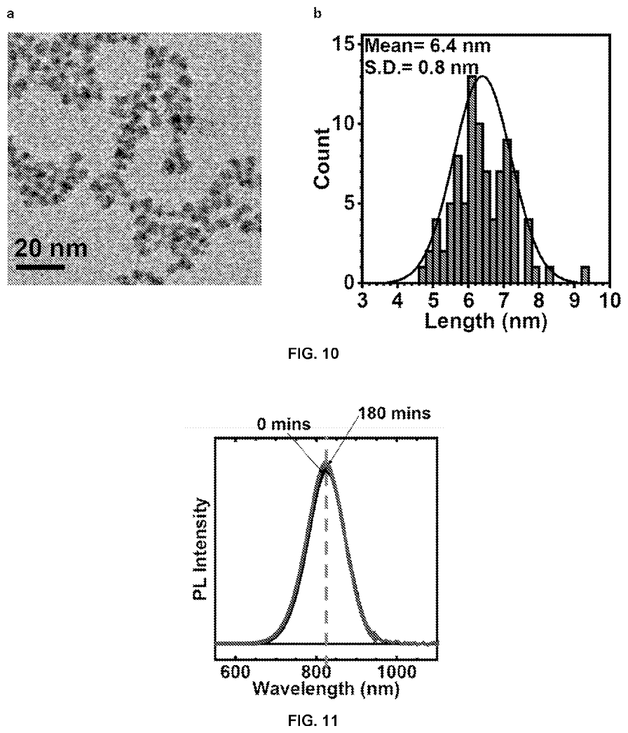

The hydrophilic QD-MSA was highly stable in water as the aqueous phase remained transparent and homogeneous without any precipitation or aggregation at room temperature for at least 1 month (FIG. 9b). DLS measurements on the ligand-exchanged In(Zn)As—In(Zn)P—GaP—ZnS QDs revealed a mean hydrodynamic size of 8.6 nm in water (FIG. 9c), slightly larger than the TEM-observed sizes (6.4 nm, FIG. 10) due to the contributions of the MSA ligand coordination sphere. No signs of larger QD agglomerates were observed in the DLS measurement, confirming their high colloidal stability in water. The average ζ potential of −15 mV also ascertained that the QD surface was covered by the negatively charged carboxylate (—COO⁻) tails of MSA ligands when dispersed in water.

The spectra in FIG. 9d show that the PL characteristics were largely preserved after ligand exchange and phase transfer, apart from a small PL blue-shift from 837 to 828 nm that could be due to a change in the dielectric environment. Although the PLQE decreased slightly from 75% to 60% after phase transfer, it is worth noting that the attenuation in PLQE is significantly less-pronounced as compared to other low-toxicity NIR QDs such as CuInS₂—ZnS (75% to 39% after phase transfer) and InAs—InP—ZnSe (32% to 21% after phase transfer) (Xia, C. et al., Chem. Mater. 2017, 29, 4940-4951; and Xie, R. et al, Nano Res. 2008, 1, 457-464). We attributed the good retention of PL performance to the thick passivating shell layers, which functioned to reduce the occurrence of defects that would otherwise promote non-radiative recombination pathways. The NIR PLQE of 60% in water also far exceeds the performance of other commercially available NIR dyes such as indocyanine green (13% at 818 nm) and IR-140 (17% at 843 nm) (Rurack, K. & Spieles, M., Anal. Chem. 2011, 83, 1232-1242). We further tested the photo-stability of the MSA-capped QDs and observed a negligible 1% attenuation in PL intensity (FIG. 9e) and no change in spectral characteristics (FIG. 11) after three hours of 405 nm (30 mW) laser photo-excitation under ambient conditions, thereby demonstrating robust photo-stability for its intended bioimaging applications.

TABLE 2
Table summarizing the calculated PL lifetime parameters
of QD-MSA. The values of Γᵣ and Γₙᵣ were calculated
using equation (5) shown above.

| Sample | PLQE/% | τ/ns | Γᵣ/μs⁻¹ | Γₙᵣ/μs⁻¹ |
|---|---|---|---|---|
| QD-MSA | 59.9 | 56.0 | 10.7 | 7.2 |

### Example 11. In Vitro Studies of QD-MSA

To further validate the applicability of our NIR QDs for bioimaging applications, we performed an in vitro study on human cervical carcinoma HeLa cells using fluorescence confocal microscopy.

HeLa Cell Culture and Confocal Imaging

HeLa cells were cultured in growth media (RPMI 1640) maintained at 37° C. with a humidified atmosphere of 5%_{CO}_{₂}. Cellular imaging of the QD were visualized by confocal microscopy. HeLa cells were seeded onto coverslips in 6-well culture plates at a density of 100×10³ cells per well in growth media. After 24 hours, the culture media were replaced with fresh RPMI containing 0.5 mg/mL or 1.0 mg/mL of the QD-MSA, and incubated for another 24 hours. Thereafter, the QD-containing media were aspirated, and the cells were stained with 1 μg/mL Hoechst (nuclear dye) and 500 nM Mito-Tracker Green (mitochondria dye) in RPMI for 15 minutes each in the incubator. The cells were then rinsed twice with PBS and fixed with 2% PFA for 5 mins at RT. After another round of rinsing with PBS, the cells on the coverslips were mounted onto microscope slides and imaged using a confocal microscope (Olympus FV1000) with a 405 nm laser excitation (1.5 mW) for Hoechst and QDs, and 488 nm (2.4 mW) for MitoTracker Green. Confocal images were collected in 3 bandpass detector channels: blue (430-470 nm), green (505-525 nm) and red (>650 nm).

MTT Cell Viability Assay

The MTT cell viability assay was used to determine the cytotoxicity of the QD on HeLa cells. Briefly, an MTT stock solution was prepared by dissolving MTT in PBS at 5 mg/mL, filter-sterilized and stored away from light at 4° C. until further use. Cells were seeded on clear, flat-bottom 96-well culture plates at a density of 6×10³ cells in 100 μL of growth media per well. After 24 hours, the culture media were replaced with 100 μL/well of fresh RPMI containing varying concentrations of the QD (15 μg mL⁻¹ to 1 mg mL⁻¹) and incubated for another 24 hours. Thereafter, the QD-containing media were aspirated and replenished with 100 μL/well of MTT solution (diluted to 0.5 mg/mL with RPMI) and incubated for 50 minutes. The MTT solution was then aspirated and DMSO (100 μL/well) was added to dissolve the dark blue crystals formed. Cell viability was determined by measuring the absorbance at 570 nm using a microplate reader and expressed as a percentage of the non-treated control wells.

### Results and Discussion

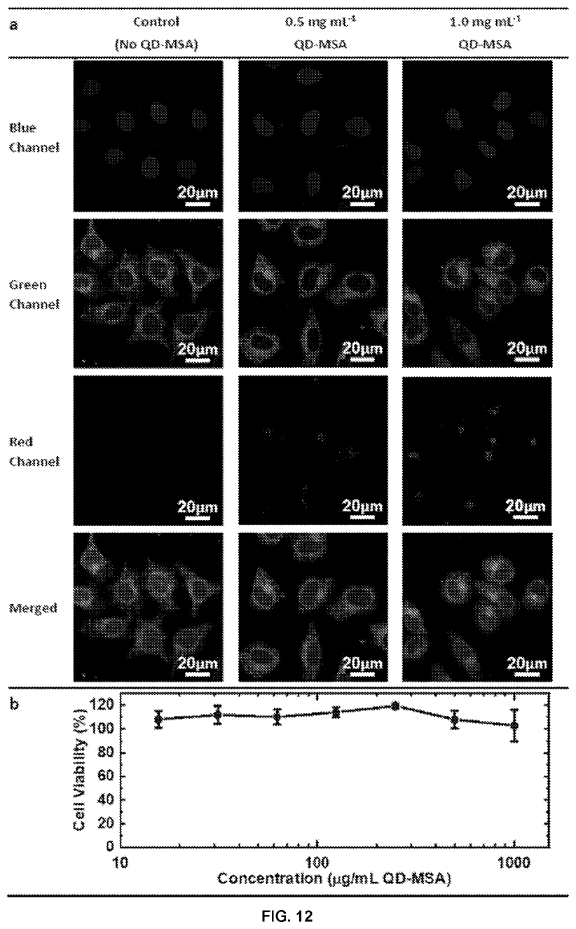

The MSA-capped hydrophilic NIR QDs in water were incubated with HeLa cells (0.5 mg mL⁻¹ and 1 mg mL⁻¹ QD-MSA in RPMI 1640 medium) for 24 hours before being imaged by a fluorescence confocal microscope with 405 nm laser excitation for Hoechst and the QDs, and with 488 nm laser excitation for MitoTracker Green. The visualized HeLa cells appeared brightly-luminescent under laser excitation, clearly demarcating the Hoechst-stained nucleus (blue), MitoTracker Green-stained mitochondria (green) and the NIR QDs (red) in FIG. 12a. We also obtained images of HeLa cells without QD-MSA incubation as a control, and the absence of any NIR fluorescence in the red channel ascertained that the NIR luminescence was due to the QD-MSA and not due to cellular auto-fluorescence. It was noted that the high-contrast fluorescence images were obtained under low laser excitation of 1.5 mW due to the high PLQE of the NIR QD-MSA in water. The merged fluorescence images and staining pattern indicated that our water-soluble QD-MSA localized primarily within the cytoplasmic space, presumably in endosomal bodies as reported for other non-functionalized nanomaterials (Mao, Z., Zhou, X. & Gao, C., Biomater. Sci. 2013, 1, 896-911). We further noted that the carboxylate (—COO⁻) tails of the MSA ligands can be readily functionalized with other bioconjugation agents such as folic acid and other cancer specific antibodies to promote receptor-mediated selective uptake in specific cellular organelles for targeted bioimaging and tagging (Quarta, A. et al., Langmuir 2009, 25, 12614-12622; Yong, K.-T. et al., ACS Nano 2009, 3, 502-510; and Bharali, D. J. et al., J. Am. Chem. Soc. 2005, 127, 11364-11371). Our QDs also exhibited low cytotoxicity (FIG. 12b), with over 98% cell viability after 24 hours of incubation with the NIR QD-MSA at varying concentrations from 15 μg mL⁻¹ to 1 mg mL⁻¹.

Taken together, the high PLQE and strong stability under physiological conditions (described in Example 10), coupled with low cytotoxicity, validated our MSA-capped NIR QD dye as an outstanding candidate for high-contrast bioimaging across different platforms from deep tissues to in vitro and in vivo applications. In combination with the deep imaging performance of our artificial-neural-network-enhanced laser-scanning imaging technique, this could become a powerful research platform for advancing the discovery of drugs for immunotherapy and targeted therapy in cancer.

1. A computerized method for processing scattered images obtained by imaging through scattering media, comprising:
providing a trained neural network model trained with a training dataset of scattered images comprising associated pairs of low-resolution images and high-resolution images, each image comprising a series of separated bands;
receiving an input scattered image by the trained neural network model;
processing the input scattered image using the trained neural network model; and
generating an output image by the trained neural network model in response to said processing of the input scattered image,
wherein the output image has a higher resolution than the input scattered image.

2. The computerized method according to claim 1, further comprising training the neural network model, said training comprising:
(a) extracting, from the training dataset, an associated pair of low-resolution and high-resolution images;
(b) identifying an input pixel from the low-resolution image and a corresponding true output pixel from the high-resolution image;
(c) selecting a cluster of pixels from the low-resolution image, the cluster of pixels surrounding the input pixel;
(d) weighting each pixel in a cluster of pixels using a set of weight and bias parameters;
(e) generating a processed output pixel from said processing of the cluster of pixels;
(f) determining an error between the processed output pixel and the true output pixel;
(g) backpropagating the error to adjust the parameters in step (d);
(h) iteratively performing steps (d) to (g) to minimize the error,
wherein the minimized error is associated with an optimized set of parameters for the neural network model.

3. The computerized method according to claim 2, said training further comprising repeating steps (a) to (h) for each associated pair of images in the training dataset.

4. The computerized method according to claim 2, wherein step (d) comprises successively weighting each pixel at least twice.

5. The computerized method according to claim 2, wherein step (e) comprises:
generating a raw output pixel from said processing of the cluster of pixels;
processing the raw output pixel using a logistic function; and
generating the processed output pixel from said processing of the raw output pixel.

6. The computerized method according to claim 2, wherein step (g) is performed using a gradient descent function.

7. The computerized method according to claim 2, wherein the error comprises a mean squared error.

8. The computerized method according to claim 2, wherein the scattered images are fluorescence images.

9. A method of imaging a part or the whole of a human or animal body, using an imaging device, comprising:
a near-infrared light source;
a light directing means or apparatus;
an array comprising nanocrystals capable of fluorescing upon excitation from light from the near-infrared light source; and
a detecting means or apparatus configured to detect light emitted by the nanocrystals, where the method comprises the steps of:
(a) positioning a first side of the part or whole of the human or animal body to be imaged to face the near-infrared light source, light directing means or apparatus and a detecting means or apparatus and a second side of the part or whole of the human or animal body to be imaged to face an array comprising nanocrystals;
(b) directing near-infrared light from the near-infrared light source through the first and second surfaces of the part or whole of the human or animal body to be imaged via the light directing means or apparatus and into the array comprising nanocrystals; and
(c) detecting fluorescent light released from the nanocrystals using the detecting means or apparatus.

10. The method according to claim 9, further comprising the step of capturing an image of the part or whole of the human or animal body to be imaged based on the detected fluorescent light, the image being a scattered image.

11. The method according to claim 10, further comprising the step of processing the scattered image using the computerized method to enhance the scattered image.

12. The method according to claim 9, wherein, in the imaging device one or more of the following apply:
(a) the near-infrared light source is a laser capable of emitting light at near-infrared wavelengths;
(b) the light directing means or apparatus comprises a mirror;
(c) the array comprising giant shell quantum dots is positioned on a moveable platform such that one or both of the following apply: the array is movable relative to the near-infrared light source; and a light beam from the near-infrared light source is moveable relative to the array; and
(d) the detecting means or apparatus further comprises an imaging apparatus.

13. The method according to claim 9, wherein, in the imaging device the nanocrystals capable of fluorescing upon excitation from light from the near-infrared light source are giant shell quantum dots having the formula:
In(Zn)As—In(Zn)P—GaP—ZnS
wherein:
In(Zn)As is the core of the quantum dot;
In(Zn)P is the giant shell;
GaP represents an interlayer shell between In(Zn)P and ZnS; and
ZnS represents an outer layer shell of the quantum dot.

14. The method according to claim 13, wherein the quantum dot is one in which one or more of the following apply:
(a) the ZnS outer layer comprises ZnS and a hydrophobic or a hydrophilic organic compound;
(b) the quantum dot displays an emission peak at from 820 to 850 nm;
the quantum dot displays a photoluminescence lifetime of from 20 to 100 ns;
the quantum dot absorbs light at a wavelength of from 400 to 800 nm;
the quantum dot displays a photoluminescence quantum efficiency of from 60 to 75%;
(c) the quantum dot has an average size according to transmission electron microscopy of from 6 to 7 nm;
the quantum dot has an average hydrodynamic size of from 8 to 9 nm, such as 8.6 nm; and
(d) the atomic percentages in the quantum dot are as follows: In from 35 to 45%; As from 1 to 5%; P from 25 to 35%; Zn from 5 to 10%; Ga from 5 to 9%; and S from 8 to 15%.

15. An imaging device comprising:
a near-infrared light source;
a light directing means or apparatus;
an array comprising nanocrystals that are capable of fluorescing upon excitation from light from the near-infrared light source; and
a detecting means or apparatus configured to detect light emitted by the nanocrystals.

16. The imaging device according to claim 15, wherein one or more of the following apply:
(a) the near-infrared light source is a laser capable of emitting light at near-infrared wavelengths;
(b) the light directing means of apparatus comprises a mirror; and
(c) the array comprising nanocrystals is positioned on a moveable platform such that one or both of the following apply: the array is movable relative to the near-infrared light source and a light beam from the near-infrared light source is moveable relative to the array.

16. (canceled)

17. The imaging device according to claim 15, wherein, in the imaging device the nanocrystals capable of fluorescing upon excitation from light from the near-infrared light source are giant shell quantum dots having the formula:
In(Zn)As—In(Zn)P—GaP—ZnS
wherein:
In(Zn)As is the core of the quantum dot;
In(Zn)P is the giant shell;
GaP represents an interlayer shell between In(Zn)P and ZnS; and
ZnS represents an outer layer shell of the quantum dot.

18. The imaging device according to claim 17, wherein the quantum dot is one in which one or more of the following apply:
(a) the ZnS outer layer comprises ZnS and a hydrophobic or a hydrophilic organic compound;
(b) the quantum dot displays an emission peak at from 820 to 850 nm;
the quantum dot displays a photoluminescence lifetime of from 20 to 100 ns;
the quantum dot absorbs light at a wavelength of from 400 to 800 nm;
the quantum dot displays a photoluminescence quantum efficiency of from 60 to 75%;
(c) the quantum dot has an average size according to transmission electron microscopy of from 6 to 7 nm;
the quantum dot has an average hydrodynamic size of from 8 to 9 nm; and
(d) the atomic percentages in the quantum dot are as follows: In from 35 to 45%; As from 1 to 5%; P from 25 to 35%; Zn from 5 to 10%; Ga from 5 to 9%; and S from 8 to 15%.

19. A method of diagnosis comprising the steps of:
(a) supplying a plurality of nanocrystals capable of fluorescing upon excitation from light from the near-infrared light source to a subject;
(b) subjecting a target site on the subject to light irradiation; and
(c) detecting a signal, or lack thereof, generated by the nanocrystals to provide a diagnosis.

20. The method according to claim 19, further comprising the step of capturing an image of the target site based on the detected signal, the image being a scattered image.

21. (canceled)

22. (canceled)

23. (canceled)
